(19) United States
(12) Patent Application Publication
(10) Pub. No.: US 2010/0049199 A1
(43) Pub. Date: Feb. 25, 2010

(54) TIBIAL GUIDE FOR ACL REPAIR HAVING MOVEABLE DISTAL FEATURES

(75) Inventor: Paul Re, Boston, MA (US)

Correspondence Address: Mark Farber, Esq.;UNITED STATES SURGICAL,, a Division of Tyco Healthcare Group LP, 150 Glover Avenue, Norwalk, CT, 06856, US

(73) Assignee: Tyco Healthcare Group LP

(21) Appl. No.: 12/548,692

(22) Filed: Aug. 27, 2009

Related U.S. Application Data
(63) Continuation-in-part of application No. 12/367,007, filed on Feb. 6, 2009.
(60) Provisional application No. 61/066,572, filed on Feb. 21, 2008.

(51) Int. Cl. A61B 17/58

(52) U.S. Cl. 606/88

(57) ABSTRACT

A device for positioning a tibial tunnel during ACL reconstruction is provided. The device may include an elongated body having proximal and distal ends, an arm extending from the distal end of the elongated body. A distal portion of the arm may be configured for insertion into a preformed opening in a femur. The arm and the body may be moveable relative to each other. Also, the device may include an additional member that is moveable relative to the arm and the body.

### CROSS REFERENCE TO RELATED APPLICATIONS

This application is a continuation-in part of, and claims the benefit of priority to, U.S. patent application Ser. No. 12/367,007, filed Feb. 6, 2009, entitled “Device for Orienting the Tibial Tunnel Position During an ACL Reconstruction” and U.S. Provisional Patent Application Ser. No. 61/066,572, filed Feb. 21, 2008, entitled “Device for Orienting the Tibial Tunnel Position During an ACL Reconstruction,” the disclosures of each being incorporated herein by reference in their entirety. In addition, this application is related to U.S. Provisional Patent Application Ser. No. 61/066,575, filed Feb. 21, 2008, entitled “Guide for Creating a Femoral Tunnel During an ACL Reconstruction” and U.S. patent application Ser. No. 12/366,967, filed Feb. 6, 2009, entitled “Guide for Creating a Femoral Tunnel During an ACL Reconstruction,” the disclosures of each also being incorporated herein by reference in their entirety.

### BACKGROUND

1. Technical Field

This invention relates to surgical apparatus and procedures in general, and more particularly to surgical apparatus and procedures for reconstructing a ligament.

2. Background of Related Art

A ligament is a piece of fibrous tissue which connects one bone to another. Ligaments are frequently damaged (e.g., detached or torn or ruptured, etc.) as the result of injury and/or accident. A damaged ligament can cause instability, impede proper motion of a joint and cause pain. Various procedures have been developed to repair or replace a damaged ligament. The specific procedure used depends on the particular ligament which is to be restored and on the extent of the damage.

One ligament which is frequently damaged as the result of injury and/or accident is the anterior cruciate ligament (i.e., the ACL). Looking first at FIGS. 1 and 2, it will be seen that the ACL 5 extends between the top of the tibia 10 and the bottom of the femur 15. A damaged ACL can cause instability of the knee joint and cause substantial pain and arthritis. For this reason, ACL reconstruction is a common procedure with more than 100,000 cases being performed in the United States annually.

Various procedures have been developed to restore and/or reconstruct a damaged ACL through a graft ligament replacement. Traditionally, this procedure is performed utilizing a trans-tibial approach. In this approach, a bone tunnel 20 (FIG. 3) is first drilled up through tibia 10. Tibial tunnel 20 is then used access the interior of the knee joint, and it is from tibial tunnel 20 that the position of a femoral tunnel 25 is determined. In this respect, it should be appreciated that the proper positioning of femoral tunnel 25 is important and that numerous guides have been designed to ensure that tibial tunnel 20 is correctly positioned in order to properly position the resulting femoral tunnel 25.

Looking next at FIGS. 4, 5 and 6, simple tibial tunnel positioning guides generally consist of a hooked tip that may be positioned along the ACL footprint on the tibia at a position chosen by the surgeon. Other tibial tunnel positioning guides are more constraining, in order to attempt to obtain a more reliable and reproducible position for the tibial tunnel. As shown in FIG. 7, some other tibial tunnel positioning guides reference the tibial base of the posterior cruciate ligament (“PCL”) (U.S. Pat. No. 5,409,494 to Morgan et al.).

Looking next at FIG. 8, still another guide references the roof of the intercondylar notch, as well as orienting the guide's position relative to the plane of the tibial plateau (U.S. Pat. No. 6,254,605, by Howell et al.). This referencing is done in an attempt to avoid impingement of the femoral roof by the graft ligament.

All of these prior art tibial tunnel positioning guides, while utilizing different referencing points and methods, still share the same overall approach: each of these guides is used to orient the tibial tunnel first, but in a position deemed appropriate for the femoral tunnel, which is thereafter drilled through that tibial tunnel. The limitations of such an approach is that the position of the tibial tunnel is often compromised in order to later drill an appropriate femoral tunnel. This often results in the tibial tunnel being placed in a position which is more posterior and more vertical than is anatomically desired.

Proper placement of the femoral tunnel is imperative in order for the ACL graft to be properly positioned on the femur. However, as a result of using the aforementioned trans-tibial technique, the position of the femoral tunnel is effectively dictated by the position of the first-drilled tibial tunnel. This often results in a femoral tunnel position, and thus, an ACL reconstruction (i.e., graft orientation, etc.) that is less than optimal.

In an attempt to better position the femoral tunnel, surgeons have recently begun utilizing the so-called “medial portal technique” to drill and create the femoral tunnel. An embodiment of a femoral drill guide for use in medial portal techniques is described in commonly owned patent application Ser. No. 12/366,967, the content of which are incorporated by reference in its entirety, and is shown generally as femoral guide 100 in FIG. 4. By drilling the femoral tunnel through the medial portal or an accessory portal, the femoral and tibial tunnels may be drilled independently of one another and, therefore, in a more appropriate anatomical position. While the medial portal approach greatly improves the ability of the surgeon to more accurately position the femoral tunnel, the older, simple trans-tibial guides are still used by the surgeon to position the tibial tunnel.

Therefore, it would be beneficial to have a device and method for orienting the position of a second-drilled tibial tunnel based on a first-drilled femoral tunnel. It would further be beneficial to have a device and method for positioning a tibial tunnel utilizing the medial portal approach prior to drilling a femoral tunnel.

### SUMMARY

A device for positioning a tibial tunnel during ACL reconstruction is provided. The device includes a portion insertable into a pre-formed opening in the femur. The device may further include an elongated body having proximal and distal ends and an arm extending at an angle from the distal end of the elongated body, the arm being configured for insertion through a medial portal. The portion insertable into a pre-formed opening in the femur may include a tip formed on a distal end of the arm.

The elongated body of the positioning device may be arced. The arm may be configured to point to the position of the resulting tibial tunnel on a tibial plateau when the distal tip is disposed in a femoral tunnel. The arm may include a pointed elbow configured to point to the position of the resulting tibial tunnel on the tibial plateau/ACL footprint. The arm may be configured to orient the angle of the resulting graft in the sagittal plane. The arm may extend from elongated body at an angle from about fifty degrees (50°) to about sixty degrees (60°). The angle between the elongated body and the arm may be adjustable. The arm may include a lateral projection. The proximal end of the elongated body may be configured for connection to an outrigger. The outrigger may be configured to direct a guide wire through the tibial. Also provided is a method for positioning a tibial tunnel during ACL reconstruction. The method includes the steps of forming an opening in a femur bone, inserting a portion of a device into the opening, and using the device to position an opening in a tibia bone. The step of creating an opening in a femur bone may performed using a medial portal approach. The device may include an elongated body, an arm extending at an angle from a distal end of the elongated body, and a tip formed on a distal end of the arm, the tip being configured for insertion into the femoral tunnel. The method may further include the step of positioning the device by referencing at least one of a lateral wall of the femoral notch and one or more tibial spines.

The device may further include a lateral projection for referencing the femoral notch. The method may further include the step of adjusting the coronal medial/lateral orientation angle of the arm of the device in a way that mimics an intact ACL. The arm of the device may be configured for insertion through a medial portal. The method may further include the step of flexing the knee through a range of motion to check for resultant graft impingement. A proximal end of the arm may include an elbow for engaging the tibia.

Additional provided is a method for positioning a tibial tunnel during ACL reconstruction. The method includes the steps of providing a tibial guide including an elongated body, an arm extending at an angle from a distal end of the elongated body, and a tip formed on a distal end of the arm, the tip including a point for engaging a femur, inserting the distal end of the elongated body into a knee joint using a medial portal approach, engaging the pointed tip with the femur in a position corresponding to that of a desired femoral tunnel, and positioning the tibial guide by referencing at least one of a lateral wall of the femoral notch and one or more tibial spines.

The present invention, in accordance with various embodiments thereof, may provide a device for positioning a tibial tunnel during ACL reconstruction, the device comprising: an elongated body having proximal and distal ends; and an arm extending from the distal end of the elongated body, a distal portion of the arm being configured for insertion into a pre-formed opening in a femur, wherein the arm and the body are moveable relative to each other.

The arm and the body may be moveable, e.g., pivotable, such that an angle between the elongated body and the arm is adjustable. The arm and the body may be pivotable relative to each other between a first position, in which the arm and the body are generally aligned relative to each other, and a second position, in which the arm and the body are misaligned relative to each other. In the second position, the arm and the body may be disposed at an angle of about fifty degrees (50°) to about sixty degrees (60°). At least one of the arm and the body may include a physical stop for preventing the arm from being over-pivoted relative to the body.

At least one of the arm and the body may include one of a groove, a knurl, a protrusion and a detent for providing an indication to a user that the arm and the body are in a particular orientation relative to each other. The device may also include an actuating member for moving the body and the arm relative to each other. The arm may be moveable in its entirety relative to the body, or a portion of the arm may be moveable relative to the body. Advantageously, the portion of the arm that is moveable relative to the body is a pointing end that is configured to point to the position of a tibial tunnel on a tibial plateau when a distal tip of the arm is disposed in an opening in a femur.

The present invention, in accordance with various embodiments thereof, may also provide a device for positioning a tibial tunnel during ACL reconstruction, the device comprising: an elongated body having proximal and distal ends; and an arm extending from the distal end of the elongated body, a distal portion of the arm being configured for insertion into a pre-formed opening in a femur, and a member which is moveable relative to the arm and the body.

The arm and the body may be at least one of integrally formed and rigidly connected to each other. The member may be a tibial pointing tip which is pivotable relative to the arm and the body. In an embodiment, the tibial pointing tip may be pivotable relative to the arm and the body between a first position, in which the tibial pointing tip is generally aligned with the body, and a second position, in which the tibial pointing tip is misaligned relative to the body. In another embodiment, the tibial pointing tip may be pivotable relative to the arm and the body between a first position, in which the tibial pointing tip is generally aligned with the arm, and a second position, in which the tibial pointing tip is misaligned relative to the arm. In the second position, the tibial pointing tip and the body may be disposed at an angle of about fifty degrees (50°) to about sixty degrees (60°) relative to each other.

At least one of the tibial pointing tip, the arm and the body may include a physical stop for preventing the tibial pointing tip from being over-pivoted relative to the body. Also, at least one of the tibial pointing tip, the arm and the body may include one of a groove, a knurl, a protrusion and a detent for providing an indication to a user that the tibial pointing tip and the body are in a particular orientation relative to each other. The device may also include an actuating member for moving the tibial pointing tip relative to the body. Advantageously, the tibial pointing tip is configured to point to the position of a tibial tunnel on a tibial plateau when a distal tip of the arm is disposed in an opening in the femur.

### BRIEF DESCRIPTION OF THE DRAWINGS

FIG. 1 is a perspective view of a knee joint showing an ACL;

FIG. 2 is an alternate perspective view of the knee joint of FIG. 1;

FIG. 3 is a perspective view of a knee joint including tibial and femoral tunnels (shown in phantom) and a ligament graft;

FIGS. 4-8 are views of various prior art embodiments of tibial tunnel positioning guides;

FIG. 9 is a femoral guide for use in ACL reconstruction utilizing the medial portal approach.

FIG. 10 is a side view of a tibial tunnel positioning guide according to an embodiment of the present disclosure;

FIG. 11 is a perspective view of a tibial tunnel positioning guide according to an alternative embodiment of the present disclosure;

FIG. 12 is a side view of a tibial tunnel positioning guide according to another embodiment of the present disclosure;

FIG. 13 is a side view of a tibial tunnel positioning guide according to yet another embodiment of the present disclosure;

FIG. 14 is an enlarged side view of the distal end of the tibial tunnel positioning guide of FIG. 10;

FIG. 15 is a side view of the distal end of the tibial tunnel positioning guide of FIG. 11;

FIG. 16 is an alternate side view of the distal end of the tibial tunnel positioning guide of FIGS. 11 and 15;

FIG. 17 is an end view of the distal end of the tibial tunnel positioning guide of FIGS. 11, 15 and 16;

FIG. 18 is a side view of the tibial tunnel positioning guide of FIGS. 11 and 15-17 secured to an outrigger;

FIG. 19 is partial cut away view of a knee joint including a tibial tunnel positioning guide and outrigger of FIG. 18 positioning;

FIG. 20 is a partial cut-away side view of the knee joint of FIG. 19 illustrating the path of a guide wire through the tibia;

FIG. 21 is an alternate partial cut-away side view of the knee joint of FIGS. 19 and 20;

FIG. 22 is a perspective view of a knee joint including a tibial tunnel positioning guide according to still yet another embodiment of the present disclosure and further including an outrigger.

FIG. 23 is a side view of the distal end of a tibial tunnel positioning guide in a first position, according to an example embodiment of the present invention;

FIG. 24 is a side view of the distal end of the tibial tunnel positioning guide shown in FIG. 23 in a second position;

FIG. 25 is a side view of the distal end of a tibial tunnel positioning guide in a first position, according to another example embodiment of the present invention;

FIG. 26 is a side view of the distal end of the tibial tunnel positioning guide shown in FIG. 25 in a second position; and

FIG. 27 is a side view of the distal end of a tibial tunnel positioning guide in a first position, according to another example embodiment of the present invention.

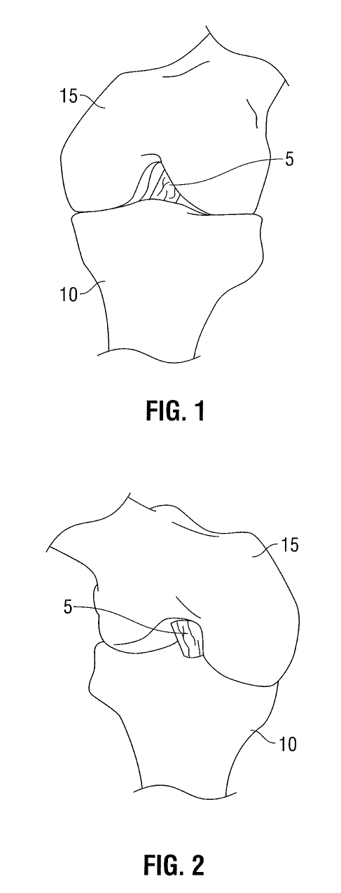

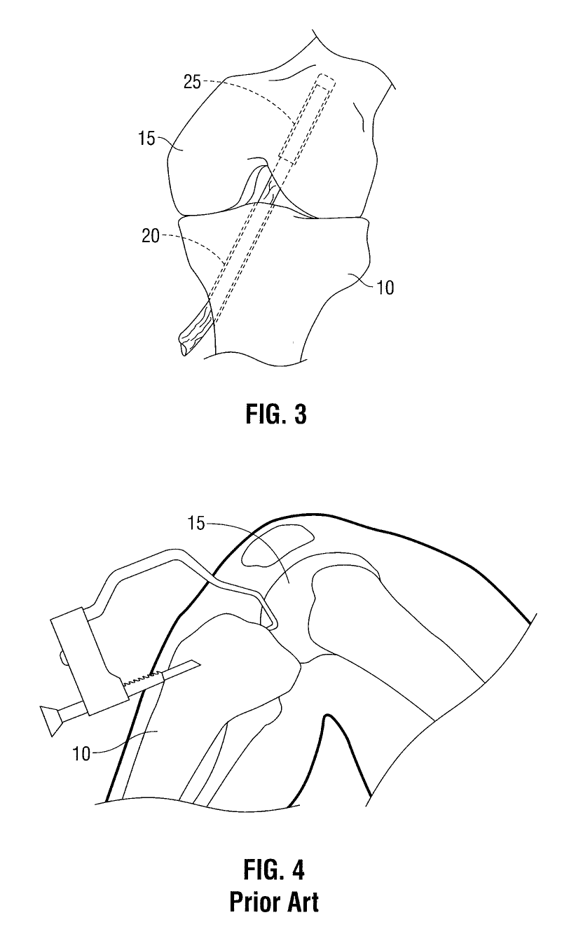

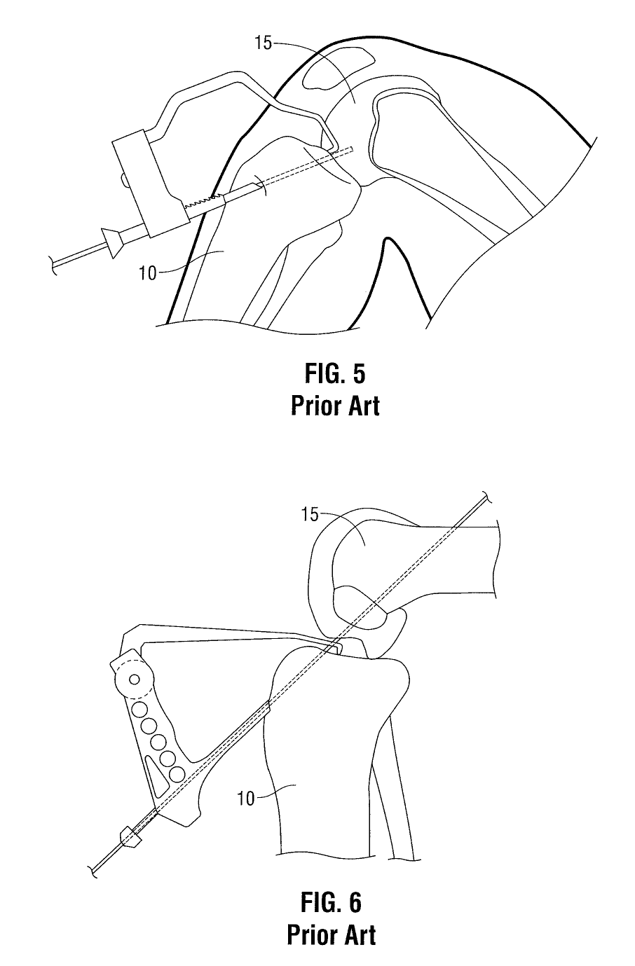

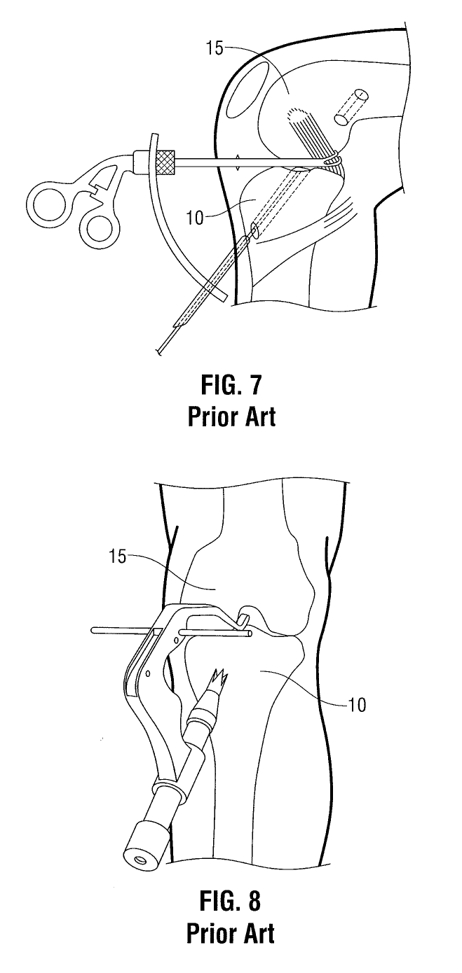

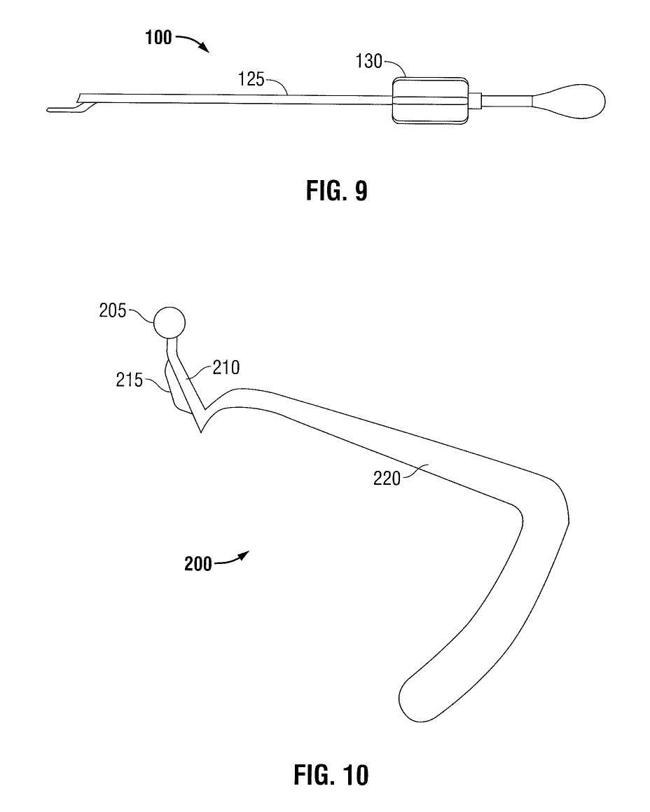

### DETAILED DESCRIPTION OF THE EMBODIMENTS

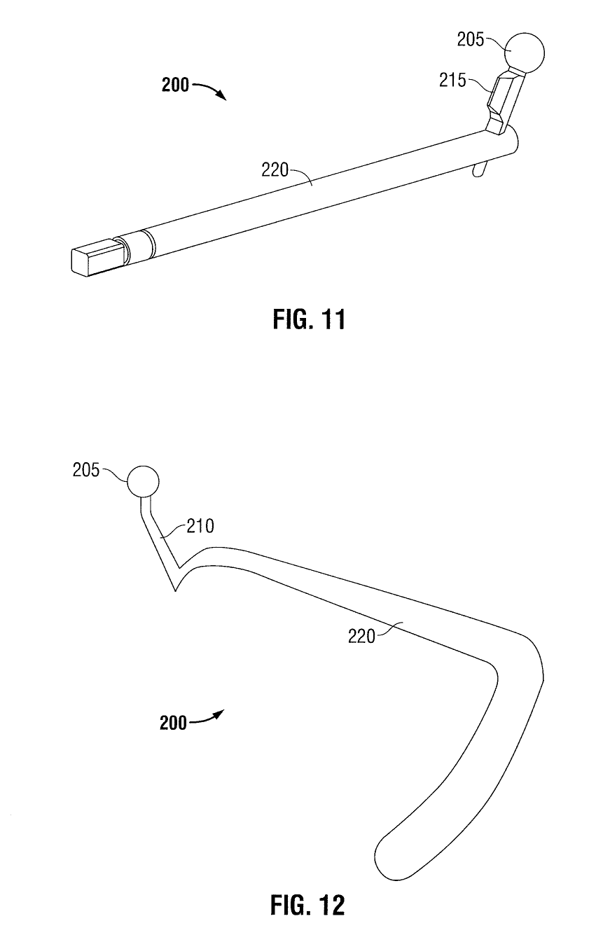

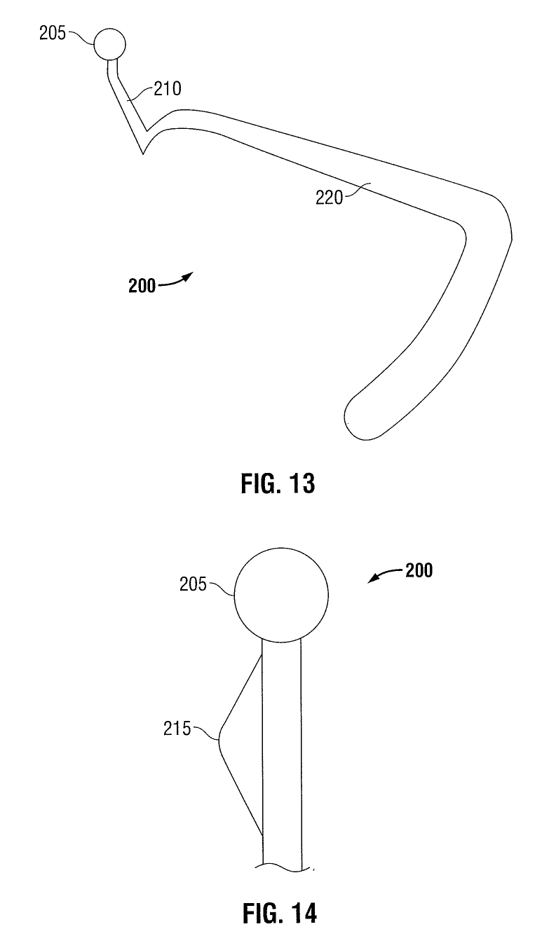

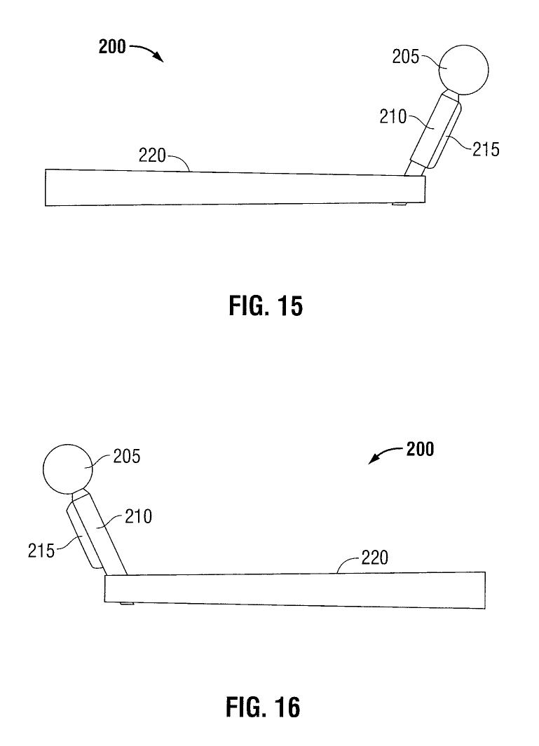

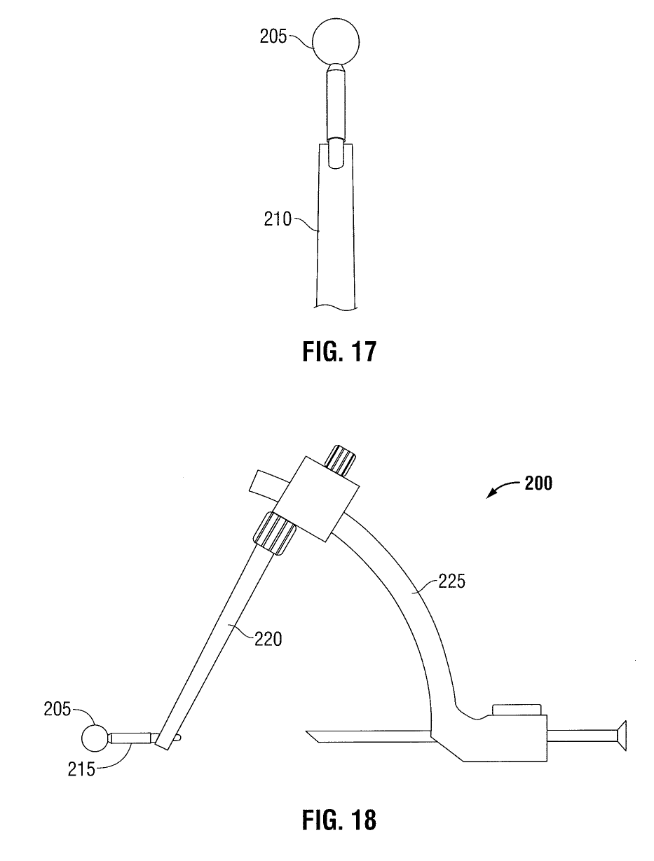

Looking now at FIGS. 10-17, there is shown a tibial tunnel positioning guide 200. Tibial tunnel positioning device 200 generally includes a distal tip 205, an arm 210 and an arced body 220. Distal tip 205 is configured to reference a previously-drilled femoral tunnel (e.g., a femoral tunnel drilled using a medial portal approach). Distal tip 205 may be configured in any shape or size suitable to mate with the femoral tunnel. As shown, distal tip 205 is generally ball-tipped and includes a diameter of substantially the size of the previously-drilled femoral tunnel. Arm 210 extends proximally from distal tip 205 and connects distal tip 205 to arced body 220. Arm 210 is configured to point to the position of the resulting tibial tunnel on the tibial plateau when distal tip 205 is disposed in femoral tunnel 25. Arm 210 is further configured to orient the angle of the resulting graft in the sagittal plane. Studies have determined that, on average, an intact ACL exists in the sagittal plane at an angle of fifty-five degrees (55°) in reference to the perpendicular axis of the tibia (or the plane of the medial or lateral surface of the tibial plateau/joint surface). Accordingly, arm 210 is configured to connect distal tip 205 to body 220 at a pre-determined angle. Arm 210 may be configured to extend from body 220 at any predetermined angle, preferably from about fifty degrees (50°) to about sixty degrees (60°). This configuration allows a surgeon to choose a particularly-angled tibial tunnel positioning guide 200 based on MRI, X-ray or other imaging data. Alternatively, tibial tunnel positioning device 200 may be configured with an angle-adjustable arm (not shown) such that arm 210 may be adjusted to any angle required to meet the needs of the surgeon.

Arm 210 may further include a lateral projection 215. Lateral projection 215 is configured to reference the lateral wall of the femoral notch to help position the resulting tibial tunnel to avoid lateral wall impingement once the graft ligament is positioned. Lateral projection 215 also aids the surgeon in orienting the medial-lateral position of tibial tunnel 20 and its orientation angle in the coronal plane. In this manner, the surgeon may set the coronal medial/lateral orientation angle of the resultant graft position in a way that mimics an intact ACL. Arm 210 may also include a pointed “elbow” which points to the resulting tibial tunnel's guide wire position on the tibial plateau/ACL footprint.

Arced body 220 extends proximally from arm 210 and is configured to facilitate insertion through the medial portal. The configuration of arced body 220 accounts for medial portal positioning to avoid the position of the portal influencing guide placement. More particularly, arm 210 of tibial tunnel positioning guide 200 may be sized and shaped to mirror the size and shape of the ligament graft to be positioned. This allows the surgeon a visual reference of what the resulting graft will look like when placed in the knee. It should be appreciated that forming arm 210 to mirror the form of the ligament graft also allows the surgeon to check for any impingement prior to drilling tibial tunnel 20. For example, once tibial tunnel positioning guide 200 is docked into the pre-drilled femoral tunnel (i.e., by placing the distal ball tip in the femoral tunnel), the surgeon may bring the knee through a range of motion to check for resultant graft impingement before creating the tibial tunnel.

Arced body 220 may also be configured for connection to an outrigger 225. (FIG. 18). Outrigger 225 positions the guide wire to be drilled through starting point of the outer tibial cortex. Arced body 220 and outrigger 225 may join at a set angle, or an adjustable angle such that the resultant outer tibial cortex starting point is not positioned too far medially, and in the position desired by the surgeon. In other words, body 220 and/or arm 210 (and therefore distal tip 205) may be set off-angle or off-axis from outrigger 225 if desired.

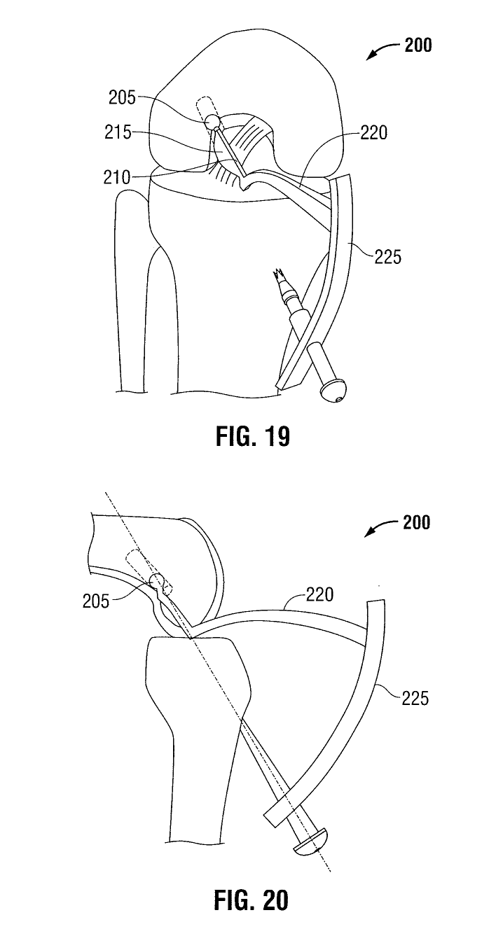

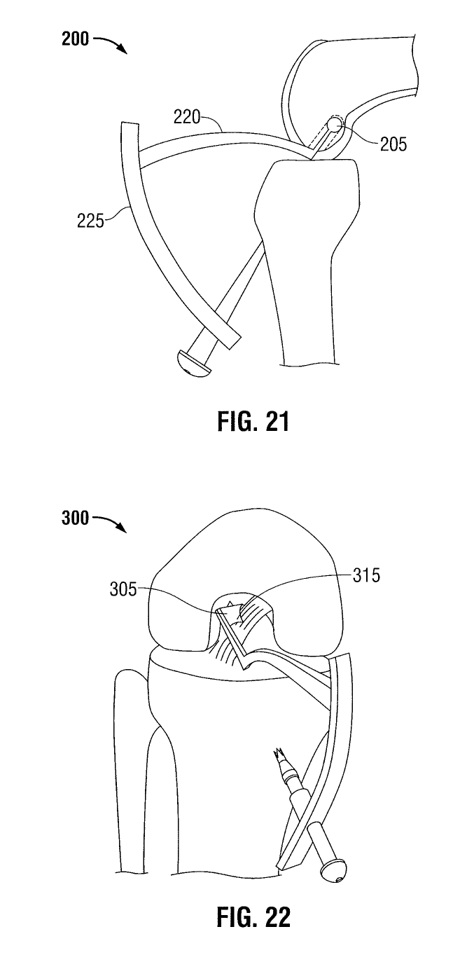

Looking next at FIGS. 19-21, tibial tunnel positioning guide 200 is placed through a medial portal with distal ball tip 205 of tibial tunnel positioning guide 200 positioned in the pre-drilled femoral tunnel. The anterior/posterior position of the resulting tibial tunnel is determined by selecting the angle of tibial tunnel positioning guide 200. The surgeon may do this in one of two ways: (i) by selecting an appropriately pre-angled guide, or (ii) by setting a desired angle on an angle-adjustable guide. The medial/lateral position of the guide (and therefore the resulting tibial tunnel) is determined by the lateral projection referencing the lateral wall of the notch. In addition, pointed elbow of arm 210 may also reference the tibial spines. In particular, the pointed elbow or arm 210 may reference the medial tibial spine to set the resultant graft in the proper anatomic coronal orientation.

Lastly, with an outrigger attached to tibial tunnel positioning guide 200, the surgeon may move the starting point of the tibial tunnel on the outer cortex, (e.g., medially and away from the MCL), if desired. With the aforementioned positions and references set, tibial tunnel positioning guide 200 is now in place so that the surgeon can confidently drill the tibial tunnel.

Looking now at FIG. 22, tibial tunnel positioning guide 300 may also be used in an approach where the femoral tunnel has not yet been drilled. In this embodiment, distal tip 305 is configured with a sharp point rather than a ball-tipped end, and a medial projection 315 rather than a lateral projection. The point of distal tip 305 and medial projection 315 are positioned referencing the location of where the PCL is inserted on the femoral notch. Tibial tunnel positioning guide may also be positioned with the point placed at any other spot along the femoral notch, or other position according to the preferences of the surgeon.

While some of the particular embodiments shown hereinabove have the body 220 and the arm 210 being integrally formed and/or rigidly connected to each other, it should be recognized that the present invention may also include other embodiments in which the body 220 and the arm 210 are not integrally formed or not rigidly connected to each other. For example, and as mentioned above, various embodiments of the present invention may include an arrangement in which the body 220 and the arm 210 are separate components that are moveable relative to each other. FIGS. 23 and 24 illustrate an example embodiment of a tibial tunnel positioning device 300 having an arrangement in which the body 220 and the arm 210 are separate components that are moveable relative to each other. Specifically, FIGS. 23 and 24 illustrate an example embodiment of the present invention having an arrangement in which the body 220 is pivotably connected to the arm 210 at a pivot point 2101, enabling the body 220 and the arm 210 to pivot relative to each other.

Providing an arrangement in which the body 220 and the arm 210 are separate components that are moveable relative to each other may provide additional advantages as compared to embodiments in which the body 220 and the arm 210 are integrally formed and/or rigidly connected to each other. For example, and as described hereinabove, in embodiments in which the body 220 and the arm 210 are integrally formed and/or rigidly connected to each other, the body 220 and the arm 210 are typically disposed at an angle relative to each other, e.g., preferably from about fifty degrees (50°) to about sixty degrees (60°). As discussed hereinabove, this angle is reflective of the function of the arm 210 in combination with, e.g., the position of the body 220 relative to the medial portal. More specifically, this angle reflects that, when the device is in place within a patient such that the body 220 is disposed within the medial portal formed by the surgeon, the arm 210 advantageously is configured to point to the position of the resulting tibial tunnel on the tibial plateau when distal tip 205 is disposed in femoral tunnel 25, thereby orienting the angle of the resulting graft in the sagittal plane. Although having the body 220 and the arm 210 be integrally formed and/or rigidly connected to each other may provide the advantage of insuring that this angle, e.g., preferably from about fifty degrees (50°) to about sixty degrees (60°), is maintained once the device is already in position within the patient's knee, it may have the disadvantage of making it more difficult for the surgeon to initially insert the device into the patient's knee and to get the device into the desired position once inserted. This potential difficulty may manifest itself due to a surgeon's general desire to have a medial portal which is as small as possible (e.g., to promote faster healing and less pain), in combination with the fact that the anatomical structures of the knee joint may result in a relatively crowded internal surgical space. Given these anatomical constraints, when the body 220 and the arm 210 are integrally formed and/or rigidly connected to each other at an angle relative to each other of, e.g., about fifty degrees (50°) to about sixty degrees (60°), the surgeon may have difficulty fitting the device through a medial portal, and then may have further difficulty manipulating the device such that, simultaneously, 1) the distal tip 205 is positioned within the pre-formed opening in the femur, 2) the body 220 remains within the medial portal, and 3) the arm 210 points to the position of the resulting tibial tunnel on the tibial plateau. In addition, having an arrangement in which the body 220 and the arm 210 are integrally formed and/or rigidly connected to each other prevents the surgeon from adjusting the angle therebetween to accommodate variations in different patients' anatomy, e.g., different angles may be desirable for many different reasons, such as for a large patient, for a patient that has atypical PCL attachment sites, etc.

Providing an arrangement in which the body 220 and the arm 210 are separate components that are moveable relative to each other may overcome some of these difficulties. For example, in embodiments in which the body 220 and the arm 210 are separate components that are moveable relative to each other, the body 220 and the arm 210 may be moveable between various positions to facilitate their insertion and/or positioning during the surgical procedure. Referring to the example embodiment illustrated in FIGS. 23 and 24, there is shown an arrangement in which the insertion and/or positioning of the device during the surgical procedure may be facilitated by having the body 220 be pivotably connected to the arm 210 at a pivot point 2101. Referring to FIG. 23, the tibial tunnel positioning device 300 is shown in a first position, e.g., in which the body 220 and the arm 210 are pivoted to a generally straight position, such that the body 220 and the arm 210 are generally aligned with each other. This generally aligned position may enable a surgeon to more easily insert the device 300 into the medial portal and to place the device into an advantageous position prior to its final positioning. Once the surgeon has inserted the device 300 through the medial portal and placed the device into a generally desired position, the surgeon may then cause the device 300 to move to a second position, such as the position illustrated in FIG. 24. As shown in FIG. 24, in the second position, the tibial tunnel positioning device 300 is no longer straight or aligned but rather the body 220 and the arm 210 are pivoted to an angled position relative to each other. This angled position is preferably from about fifty degrees (50°) to about sixty degrees (60°), such that the arm 210 may accomplish the above-referenced objectives that, when the device is in place within a patient such that the body 220 is disposed within the medial portal formed by the surgeon, the arm 210 points to the position of the resulting tibial tunnel on the tibial plateau when distal tip 205 is disposed in femoral tunnel 25, thereby orienting the angle of the resulting graft in the sagittal plane. By enabling a surgeon to form the angle between the body 220 and the arm 210 after the device is already inserted into the medial portal and after the device has already been placed into an advantageous position, the surgeon may have relatively less difficulty fitting the device through the medial portal, and then manipulating the device such that, simultaneously, 1) the distal tip 205 is positioned within the pre-formed opening in the femur, 2) the body 220 remains within the medial portal, and 3) the arm 210 points to the position of the resulting tibial tunnel on the tibial plateau. In addition, having an arrangement in which the body 220 and the arm 210 are moveable, e.g., pivotable, relative to each other may enable a surgeon to adjust the angle to accommodate variations in different patients' anatomy.

As set forth above, once the surgeon has inserted the device 300 through the medial portal and placed the device into a generally desired position, the surgeon may then cause the device 300 to move to a second position, such as the position illustrated in FIG. 24. There are various ways in which the surgeon may cause the device to move between the first and second positions. For example, the tibial tunnel positioning device 300 may be configured such that the surgeon may utilize the anatomical structures within the knee joint to assist in moving the device 300 between the first and second positions. In such an arrangement, the surgeon may insert the device 300 through the medial portal while the device 300 is in the first, e.g., generally straight, position. The surgeon may then, while the device 300 is still in the first, e.g., generally straight, position, insert the distal tip 205 of the arm 210 into the pre-formed opening in the femur. While maintaining the distal tip 205 of the arm 210 in the pre-formed opening in the femur, the surgeon may then manipulate the body 220 so as to cause the arm 210 to pivot relative to the body 220, e.g., by placing an amount of pressure on the body 220 which is sufficient to overcome any frictional force that may exist between the arm 210 and the body 220. The surgeon may continue to manipulate the body 220, e.g., by continuing to place pressure on the body 220, until the device 300 is in the second position, whereby the arm 210 points to the position of the resulting tibial tunnel on the tibial plateau.

While in the above-described example embodiment the tibial tunnel positioning device 300 is configured such that the surgeon may utilize the anatomical structures within the knee joint to assist in moving the device 300 between the first and second positions, the present invention also contemplates other ways in which the surgeon may cause the device to move between the first and second positions. For example, the tibial tunnel positioning device 300 may include structural features that enable a surgeon to move the device 300 between the first and second positions. In such an arrangement, an actuating member 2201, as shown schematically in FIG. 24, may extend along at least a portion of the body 220. A distal end of the actuating member 2201 may engage a portion of the arm 210. A proximal end of the actuating member 2201 may reside on a portion of the body 220 that is accessible to a surgeon when the device 200 is positioned within a patient's body. In this manner, when the device 300 is positioned within a patient's body, e.g., after the surgeon has inserted the distal tip 205 of the arm 210 into the pre-formed opening in the femur, the surgeon may manipulate the proximal end of the actuating member 2201, thereby causing the distal end of the actuating member 2201 to engage the arm 210 and move the arm 210 from its first, e.g., generally straight, position to its second, generally angled, position. It should be recognized that the actuating member 2201 shown in FIG. 24 is merely one of many types of actuating members 2201 that may be employed for the purpose of moving the device 300, and specifically the arm 210, between first and second positions.

While the tibial tunnel positioning device 300 may be configured so as to enable the arm 210 and the body 220 to freely move, e.g., freely pivot, relative to each other, it should be recognized that, in alternative embodiments, the tibial tunnel positioning device 300 may provide features that regulate, restrict or otherwise control the free movement of the arm 210 and the body 220 relative to each other. For example, the tibial tunnel positioning device 300 may provide features that limit the range of motion of the arm 210 and the body 220 relative to each other. Such a feature may include physical stops that prevent the arm 210 from being over-pivoted relative to the body 220. For example, the arm 210 and the body 220 may include one or more physical stops that contact each other when, e.g., the arm 210 and the body 220 are in a first, e.g., generally straight, position and/or when the arm 210 and the body 220 are pivoted to a second, angled position (preferably from about fifty degrees (50°) to about sixty degrees (60°)) and prevent the arm 210 and the body 220 from being pivoted beyond the desired angles relative to each other. Such physical stops may be features that extend radially from the arm 210 and/or the body 220, e.g., corresponding nubs or shoulders. Additionally or alternatively, such physical stops may simply be accomplished via the relative shapes of the arm 210 and/or body 220, e.g., the pointing tip 2102 of the arm 210 fitting within a slot or bore of the body 220, the bore or slot having a shape that prevents over-pivoting of the arm 210 when moved.

While the tibial tunnel positioning device 300 may provide features that regulate or restrict the free movement of the arm 210 and the body 220 relative to each other by limiting the range of motion of the arm 210 and the body 220 relative to each other, it should be recognized that, additionally or alternatively, the tibial tunnel positioning device 300 may provide features that regulate or restrict the free movement of the arm 210 and the body 220 relative to each other by providing resistance to the movement of the arm 210 and the body 220 relative to each other. Such features may include grooves or knurls on one or more of the arm 210 and the body 220. Such features may generate increased friction between the arm 210 and the body 220 when moved, as compared to a relatively lower friction that would be present without such features. The friction generated by such features may be relatively low, such that there is little resistance needed to move the arm 210 and the body 220 relative to each other. Alternatively, such friction may be relatively high, such that, absent a force exerted by the surgeon, the arm 210 and the body 220 will remain in their relative positions. Such features may also include one or more of protrusions and/or detents on one or more of the arm 210 and the body 220. Such protrusions and/or detents may be located at specific positions of the arm 210 and/or the body 220. For example, in an embodiment, such protrusions and/or detents are located at specific positions of the arm 210 and/or the body 220 such that a surgeon will experience a tactile indication when the arm 210 and the body 220 are at specific positions relative to each other, e.g., when they are in a first, e.g., generally straight, position and/or when the arm 210 and the body 220 are pivoted to a second, angled position. Of course, any number of features, e.g., protrusions and/or detents, indicating any number of relative positions of the arm 210 and the body shaft 220, may be employed.

In addition, the femoral tunnel positioning device 300 may provide indicia that indicate to the surgeon the position of the arm 210 and the body 220 relative to each other. Such indicia may be located at specific positions of the arm 210 and/or the body 220. For example, in an embodiment, such indicia may be located at specific positions of the arm 210 and/or the body 220 such that a surgeon will be able to ascertain the relative positions of the arm 210 and the body 220. Of course, any number of indicia, indicating any number of relative positions of the arm 210 and the body 220, may be employed. Furthermore, such indicia may be located at specific positions of the arm 210 and/or the body 220 that coincide with the specific positions of protrusions and/or detents of the arm 210 and/or the body 220, thus providing a surgeon with both a visual and a tactile indication when the arm 210 and the body 220 are at specific positions relative to each other.

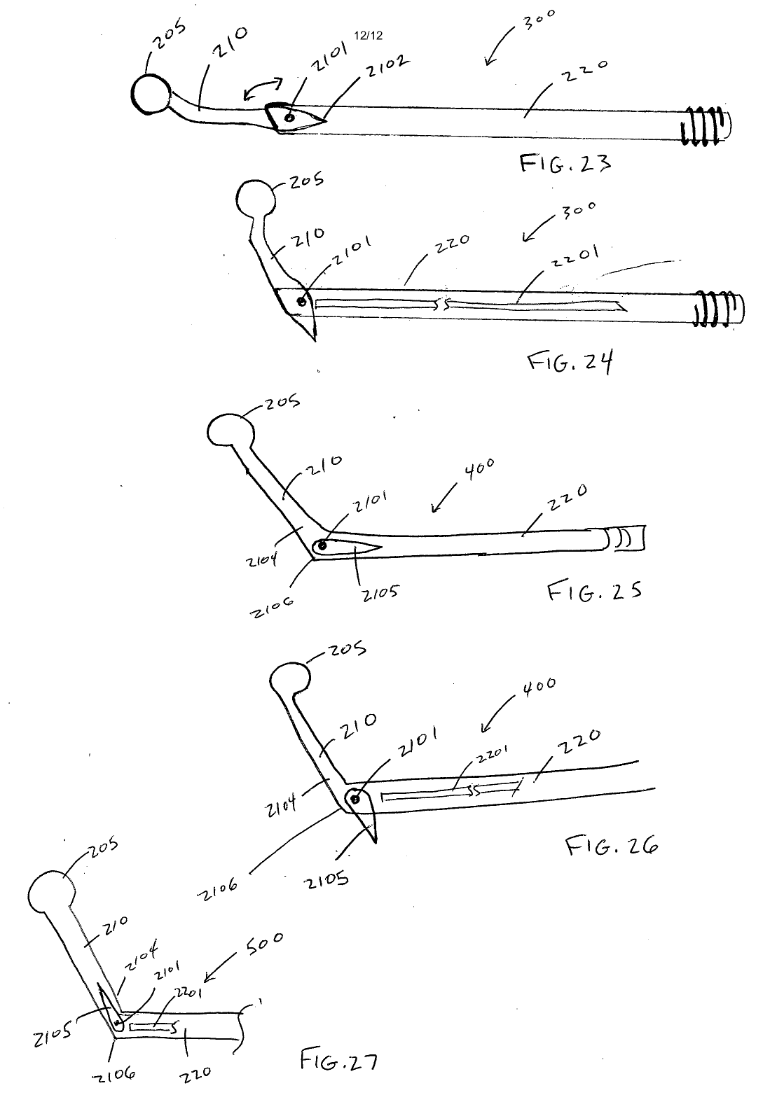

While some of the above-described embodiments of the present invention include an arrangement in which the entire arm 210 is a separate component from, and is moveable relative to, the body 220, it should be recognized that the present invention may also include other embodiments in which only a portion of the arm 210 is a separate component from, and is moveable relative to, the body 220 and the remaining portion of the arm 210. Additionally or alternatively, it should be recognized that the present invention may also include other embodiments in which a third structure, other than the body 220 and the arm 210, is a separate component from, and is moveable relative to, the body 220 and the arm 210. For example, various embodiments of the present invention may include an arrangement in which a tibial pointing tip is a separate component that is moveable relative to the arm 210 and the body 220 (a tibial pointing tip may or may not be considered a part of the arm 210—if so considered, then the tibial pointing tip may represent a portion of the arm 210 that is a separate component from, and is moveable relative to, the body 220 and the remaining portion of the arm 210; if not so considered, then the tibial pointing tip may represent a third structure that is a separate component from, and is moveable relative to, the body 220 and the arm 210). FIGS. 25, 26 and 27 illustrate example embodiments of the present invention having an arrangement in which a tibial pointing tip 2105 is a separate component that is moveable relative to at least a portion of the arm 210 and the body 220. Specifically, FIGS. 25 and 26 illustrate an example embodiment of the present invention having an arrangement in which a tibial pointing tip 2105 is pivotably connected to the device 200 at a portion 2106. The portion 2106 is a location of the device 400 at which a distal portion 2104 of the arm 210 meets the body 220. In the embodiment shown, the portion 2106 is the location at which the arm 210 is rigidly connected to the body 220 at a fixed angle, preferably from about fifty degrees (50°) to about sixty degrees (60°). The tibial pointing tip 2105 is pivotably connected to the device 400 at a pivot point 2101, enabling the tibial pointing tip 2105 to pivot relative to the body 220 and the distal portion 2104 of the arm 210.

Providing an arrangement in which a portion of the arm 210, or a third structure, is a separate component from, and is moveable relative to, the body 220 and another portion of the arm 210, may provide additional advantages as compared to embodiments in which the body 220 and the entire arm 210 are integrally formed and/or rigidly connected to each other. For example, and similar to the advantages described hereinabove in connection with the embodiments of FIGS. 23 and 24, having an arrangement in which a portion of the arm 210, or a third structure, is a separate component from, and is moveable relative to, the body 220 and another portion of the arm 210, may help a surgeon to facilitate the insertion and/or positioning of the device during the surgical procedure. Referring to the example embodiment illustrated in FIGS. 25 and 26, there is shown an arrangement in which the insertion and/or positioning of the device during the surgical procedure may be facilitated by having the tibial pointing tip 2105 be pivotably connected to the device 400 at the pivot point 2101. Referring to FIG. 25, the tibial tunnel positioning device 400 is shown in a first position, e.g., in which the tibial pointing tip 2105 is pivoted to a generally straight position relative to the body 220, such that the tibial pointing tip 2105 and the body 220 are generally aligned with each other. This generally aligned position may enable a surgeon to more easily insert the device 400 into the medial portal and to place the device into an advantageous position prior to its final positioning. Once the surgeon has inserted the device 400 through the medial portal and placed the device into a generally desired position, the surgeon may then cause the tibial pointing tip 2105 to move to a second position, such as the position illustrated in FIG. 26. As shown in FIG. 26, in the second position, the tibial pointing tip 2105 is no longer straight or aligned with body 220, but rather the body 220 and the tibial pointing tip 2105 are pivoted to an angled position relative to each other. This angled position is preferably from about fifty degrees (50°) to about sixty degrees (60°), such that the tibial pointing tip 2105 may accomplish the above-referenced objectives that, when the device is in place within a patient such that the body 220 is disposed within the medial portal formed by the surgeon, the tibial pointing tip 2105 points to the position of the resulting tibial tunnel on the tibial plateau when distal tip 205 is disposed in femoral tunnel 25. By enabling a surgeon to form the angle between the body 220 and the arm 210 after the device is already inserted into the medial portal and after the device has already been placed into an advantageous position, the surgeon may have relatively less difficulty fitting the device through the medial portal, and then manipulating the device such that, simultaneously, the distal tip 205 is positioned within the pre-formed opening in the femur, the body 220 remains within the medial portal, and the arm 210 points to the position of the resulting tibial tunnel on the tibial plateau.

Furthermore, providing an arrangement in which a portion of the arm 210, or a third structure, is a separate component from, and is moveable relative to, the body 220 and another portion of the arm 210, may provide additional advantages as compared to embodiments in which the body 220 and the entire arm 210 are moveable, e.g., pivotable, relative to each other. For example, having an arrangement in which a portion of the arm 210, or a third structure, is a separate component from, and is moveable relative to, the body 220 and another portion of the arm 210, may combine the advantages of an arrangement in which the arm 210 and the body 220 are integrally formed and/or rigidly connected to each other (e.g., insuring that the angle between the body 220 and the arm 210, e.g., preferably from about fifty degrees (50°) to about sixty degrees (60°), is maintained while the device is in position within the patient's knee) and the advantages of an arrangement in which the entire arm 210 and the body 220 are moveable, e.g., pivotable, relative to each other (e.g., helping a surgeon to facilitate the insertion and/or positioning of the device 200 during the surgical procedure).

Similar to some of the above-described embodiments, there are various ways in which the surgeon may cause a device, such as the device shown in FIGS. 25 and 26, to move between the first and second positions. For example, the tibial tunnel positioning device 400 of FIG. 25 may be configured such that the surgeon may utilize the anatomical structures within the knee joint to assist in moving the device 400 between the first and second positions. In such an arrangement, the surgeon may insert the device 400 through the medial portal while the device 400 is in the first position, e.g., wherein the tibial pointing tip 2105 is in a generally straight position relative to the body 220 such that the tibial pointing tip 2105 and the body 220 are generally aligned with each other. The surgeon may then cause a pointed end of the tibial pointing tip 2105 to contact an anatomical feature within the patient's knee. With the pointed end of the tibial pointing tip 2105 in contact with the anatomical feature within the patient's knee, a surgeon may then place an amount of pressure on the tibial pointing tip 2105 which is sufficient to overcome any frictional force that may exist between the tibial pointing tip 2105 and the body 220 so as to cause the tibial pointing tip 2105 to pivot relative to the body 220. The surgeon may continue to place such pressure on the tibial pointing tip 2105 until the device 400 is in the second position, e.g., until the tibial pointing tip 2105 is in the position shown in FIG. 26. With the tibial pointing tip so disposed, the surgeon may then insert the distal tip 205 of the arm 210 into the pre-formed opening in the femur and adjust the position of the device 200 until the pointed end of the tibial pointing tip 2105 points to the position of the resulting tibial tunnel on the tibial plateau.

Of course, it should be recognized that, while the embodiment shown in FIG. 25 provides an arrangement in which, in the first position, the tibial pointing tip 2105 is in a generally straight position relative to the body 220 such that the tibial pointing tip 2105 and the body 220 are generally aligned with each other, the present invention contemplates other embodiments in which, in the first position, the tibial pointing tip 2105 is in a generally straight position relative to the arm 210 such that the tibial pointing tip 2105 and the arm 210 are generally aligned with each other. An example of such an embodiment is shown as the tibial tunnel positioning device 500 in FIG. 27. Like previously-described embodiments, this generally aligned position of the tibial pointing tip 2105 and the arm 210 may enable a surgeon to more easily insert the device 500 into the medial portal and to place the device 500 into an advantageous position prior to its final positioning. Again, once the surgeon has inserted the device 500 through the medial portal and placed the device into a generally desired position, the surgeon may then cause the tibial pointing tip 2105 to move to a second position, in which the tibial pointing tip 2105 is no longer straight or aligned with arm 210, but rather the arm 210 and the tibial pointing tip 2105 are pivoted to an angle (preferably from about fifty degrees (50°) to about sixty degrees (60°)) relative to each other.

Similar to some of the above-described embodiments, there are various ways in which the surgeon may cause a device, such as the device shown in FIG. 27, to move between the first and second positions. For example, the tibial tunnel positioning device 500 of FIG. 27 may be configured such that the surgeon may utilize the anatomical structures within the knee joint to assist in moving the device 500 between the first and second positions. In such an arrangement, the surgeon may insert the device 500 through the medial portal while the device 500 is in the first position, e.g., wherein the tibial pointing tip 2105 is in a generally straight position relative to the arm 210 such that the tibial pointing tip 2105 and the arm 210 are generally aligned with each other. The surgeon may then cause a pointed end of the tibial pointing tip 2105 to contact an anatomical feature within the patient's knee. With the pointed end of the tibial pointing tip 2105 in contact with the anatomical feature within the patient's knee, a surgeon may then place an amount of pressure on the tibial pointing tip 2105 which is sufficient to overcome any frictional force that may exist between the tibial pointing tip 2105 and the arm 210 so as to cause the tibial pointing tip 2105 to pivot relative to the arm 210. The surgeon may continue to place such pressure on the tibial pointing tip 2105 until the device 500 is in the second position, e.g., until the tibial pointing tip 2105 is in the position shown in FIG. 26. With the tibial pointing tip so disposed, the surgeon may then insert the distal tip 205 of the arm 210 into the pre-formed opening in the femur and adjust the position of the device 500 relative to the patient's knee anatomy until the pointed end of the tibial pointing tip 2105 points to the position of the resulting tibial tunnel on the tibial plateau.

As mentioned previously, while various above-described example embodiments include arrangements in which the tibial tunnel positioning device is configured such that the surgeon may utilize the anatomical structures within the knee joint to assist in moving the device between the first and second positions, the present invention also contemplates other ways in which the surgeon may cause the device to move between the first and second positions. For example, the tibial tunnel positioning device 400 or 500 shown in FIGS. 25, 26 and 27 may include structural features that enable a surgeon to move the device 400 or 500 between the first and second positions. In such an arrangement, an actuating member 2201, as shown schematically in FIGS. 26 and 27, may extend along at least a portion of the body 220. A distal end of the actuating member 2201 may engage a portion of the tibial pointing tip 2105. A proximal end of the actuating member 2201 may reside on a portion of the body 220 that is accessible to a surgeon when the device 400 or 500 is positioned within a patient's body. In this manner, when the device 400 or 500 is positioned within a patient's body, e.g., either before or after the surgeon has inserted the distal tip 205 of the arm 210 into the pre-formed opening in the femur, the surgeon may manipulate the proximal end of the actuating member 2201, thereby causing the distal end of the actuating member 2201 to engage the tibial pointing tip 2105 and move the tibial pointing tip 2105 from its first position to its second position. It should be recognized that the actuating member 2201 shown in FIGS. 26 and 27 are merely one of many types of actuating members 2201 that may be employed for the purpose of moving the device 200, and specifically the arm tibial pointing tip 2105, between its first and second positions.

As mentioned above in connection with the embodiments described in FIGS. 23 and 24, the tibial tunnel positioning devices 400 and 500 as shown in FIGS. 25, 26 and 27 may be configured so as to enable the tibial pointing tip 2105 to freely move, e.g., freely pivot, relative to the other structures of the device, and/or the tibial tunnel positioning devices 400 and 500 may provide features that regulate, restrict or otherwise control the free movement of the tibial pointing arm 2105 relative to the other structures of the device. For example, the tibial tunnel positioning devices 400 or 500 may provide features that limit the range of motion of the tibial pointing tip 2105 relative to the other structures, such as physical stops that prevent the tibial pointing tip 2105 from being over-pivoted, e.g., corresponding nubs or shoulders, and/or via the relative shapes of the tibial pointing tip 2105 and the arm 210 and/or body 220 such as slots and bores. Additionally or alternatively, the tibial tunnel positioning devices 400 or 500 may provide features that regulate or restrict the free movement of the tibial pointing tip 2105 relative to the other structures of the device by providing resistance therebetween, e.g., grooves, knurls, protrusions, detents, etc. that generate increased friction between the tibial pointing tip 2105 and the arm 210 and/or the body 220 when moved, as compared to a relatively lower friction that would be present without such features.

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A device for positioning a tibial tunnel during ACL reconstruction, the device comprising:
an elongated body having proximal and distal ends; and
an arm extending from the distal end of the elongated body, a distal portion of the arm being configured for insertion into a pre-formed opening in a femur,
wherein the arm and the body are moveable relative to each other.

2. The device of claim 1, wherein the arm and the body are moveable such that an angle between the elongated body and the arm is adjustable.

3. The device of claim 1, wherein the arm and the body are pivotable relative to each other.

4. The device of claim 3, wherein the arm and the body are pivotable relative to each other between a first position, in which the arm and the body are generally aligned relative to each other, and a second position, in which the arm and the body are misaligned relative to each other.

5. The device of claim 4, wherein, in the second position, the arm and the body are disposed at an angle of about fifty degrees (50°) to about sixty degrees (60°).

6. The device of claim 1, wherein at least one of the arm and the body include a physical stop for preventing the arm from being over-pivoted relative to the body.

7. The device of claim 1, wherein at least one of the arm and the body include one of a groove, a knurl, a protrusion and a detent for providing an indication to a user that the arm and the body are in a particular orientation relative to each other.

8. The device of claim 1, further comprising an actuating member for moving the body and the arm relative to each other.

9. The device of claim 1, wherein the arm is moveable in its entirety relative to the body.

10. The device of claim 1, wherein a portion of the arm is moveable relative to the body.

11. The device of claim 1, wherein the portion of the arm that is moveable relative to the body is a pointing end that is configured to point to the position of a tibial tunnel on a tibial plateau when a distal tip of the arm is disposed in an opening in the femur.

12. A device for positioning a tibial tunnel during ACL reconstruction, the device comprising:
an elongated body having proximal and distal ends; and
an arm extending from the distal end of the elongated body, a distal portion of the arm being configured for insertion into a pre-formed opening in a femur,
a member which is moveable relative to the arm and the body.

13. The device of claim 21, wherein the arm and the body are at least one of integrally formed and rigidly connected to each other.

14. The device of claim 12, wherein the member is a tibial pointing tip.

15. The device of claim 14, wherein the tibial pointing tip is pivotable relative to the arm and the body.

16. The device of claim 14, wherein the tibial pointing tip is pivotable relative to the arm and the body between a first position, in which the tibial pointing tip is generally aligned with the body, and a second position, in which the tibial pointing tip is misaligned relative to the body.

17. The device of claim 14, wherein the tibial pointing tip is pivotable relative to the arm and the body between a first position, in which the tibial pointing tip is generally aligned with the arm, and a second position, in which the tibial pointing tip is misaligned relative to the arm.

18. The device of claim 14, wherein, in the second position, the tibial pointing tip and the body are disposed at an angle of about fifty degrees (50°) to about sixty degrees (60°) relative to each other.

19. The device of claim 14, wherein at least one of the tibial pointing tip, the arm and the body include a physical stop for preventing the tibial pointing tip from being over-pivoted relative to the body.

20. The device of claim 14, wherein at least one of the tibial pointing tip, the arm and the body include one of a groove, a knurl, a protrusion and a detent for providing an indication to a user that the tibial pointing tip and the body are in a particular orientation relative to each other.

21. The device of claim 14, further comprising an actuating member for moving the tibial pointing tip relative to the body.

22. The device of claim 14, wherein the tibial pointing tip is configured to point to the position of a tibial tunnel on a tibial plateau when a distal tip of the arm is disposed in an opening in the femur.
